(19) United States
(12) Patent Application Publication
(10) Pub. No.: US 2014/0296961 A1
(43) Pub. Date: Oct. 2, 2014

(54) MEDICAL IMPLANT AND METHOD FOR PRODUCTION THEREOF

(71) Applicant: BIOTRONIK AG, Buelach (CH)

(72) Inventors: Adrian Blaser, Zuerich (CH); Raimund Moehl, Forch (CH); Martin Kiekbusch, Stralsund (DE)

(21) Appl. No.: 14/189,477

(22) Filed: Feb. 25, 2014

Related U.S. Application Data
(60) Provisional application No. 61/807,349, filed on Apr. 2, 2013.

(51) Int. Cl. A61F 2/915

(52) U.S. Cl. CPC A61F 2/915
USPC 623/1.12; 623/1.16; 72/324

(57) ABSTRACT

A medical implant has a compressed and expanded state with an open-worked, preferably hollow-cylindrical main structure composed of a plurality of crosspieces, of which at least two are interconnected in each case at node elements. In the compressed state, even with complicated movements of the implant, for example when inserted into a vessel, at least one first barrier element is provided at each of a plurality of first node elements and at least one second barrier element is provided at each of a plurality of second node elements in each case adjacent to a respective first node element, such that a first barrier element and a respective adjacent second barrier element in each case lock together as the implant transitions from the expanded state into the compressed state, preferably during crimping, and thus inhibit the transition back in to the expanded state, so the barrier opposes a force in the circumferential direction and opposes a force in the radial direction. A system formed of an implant of this type and a catheter with a balloon and also a method for making such an implant and a system of this type are also described.

### PRIORITY CLAIM AND REFERENCE TO RELATED APPLICATION

The application claims priority under 35 U.S.C. §119 from prior provisional application Ser. No. 61/807,349, which was filed Apr. 2, 2013.

### FIELD

A field of the invention concerns medical implants. The invention is applicable, for example, to endovascular prostheses, endoprostheses for closing patent foramen ovale (PFO), stent graphs for treating aneurysms, endoprostheses for closing an ASD (atrial septal defect), and prostheses in the area of hard and soft tissue.

### BACKGROUND

A wide variety of medical endoprostheses or implants are known from the prior art for a wide range of applications. Particular applications include endovascular prostheses or other endoprostheses, for example stents (vessels stents (vascular stents, including of the heart and heart valve stents, for example mitral stents and pulmonary valve stents), and bile duct stents), endoprostheses for closing patent foramen ovale (PFO), stent graphs for treating aneurysms, endoprostheses for closing an ASD (atrial septal defect), and prostheses in the area of hard and soft tissue.

Stents that are used for treating stenoses (vessel constrictions) are used particularly frequently as implants. They have an open-worked, preferably hollow-cylindrical (tubular) main structure, which is open at both longitudinal ends. Here, the main structure of conventional stents is often composed of individual meshes formed from crosspieces (struts), which form zigzagged or meandering structures for example. An implant of this type is often inserted using a catheter into the vessel to be treated and is used to support the vessel over a relatively long period of time (months to years). Due to the use of stents and simultaneous or subsequent dilation, constricted areas in the vessels can be widened, thus resulting in a lumen gain.

The crosspieces of the main structure of such a stent converge in areas that will be referred to hereinafter as node elements. At least two crosspieces are interconnected at such a node element. The zigzagged or meandering structures are often interconnected in the circumferential direction by way of connecting crosspieces often extending in the longitudinal direction. Areas in which the crosspieces of a structure running in the circumferential direction adjoin one another by way of connecting crosspieces of this type and also areas in which merely crosspieces of the structure formed in the circumferential direction converge will be referred to as node elements.

Stents or other implants normally adopt two states, namely a compressed state with a small diameter and an expanded state with a larger diameter. In the compressed state, the implant can be introduced by way of a catheter into the vessel to be supported and can be positioned at the point to be treated. To this end, the implant is crimped for example onto the balloon of a catheter. The implant is then dilated at the treatment location, for example by the balloon of the catheter. Due to this change in diameter, the implant is subjected to a mechanical load during this process. Further mechanical loads of the implant may occur during the production of the implant or as the implant is moved in or with the vessel into which the implant is inserted.

Stents are also produced in part from shape-memory alloys, for example from nitinol, which can perform a temperature-dependent shape conversion. Stents of this type, often referred to as self-expandable stents, can be transferred by via temperature change, in particular a temperature increase, from the compressed state (low-temperature phase, martensite) into the expanded state (high-temperature phase, austenite). Body temperature after implant can be relied upon to achieve the expanded state.

Self-expandable systems currently offered on the market formed of a self-expandable stent and a catheter often include a tubular outer sleeve, which is slid as an expansion barrier over the stent arranged on the catheter and fixes said stent in the compressed state. To release the stent in the body, this protective sleeve is retracted. A disadvantage of this outer sleeve is the increased diameter of the system, in particular in the distal area, due to the presence of an additional layer.

Furthermore, such an outer sleeve in conjunction with a curved stent has a comparatively high flexural rigidity, which noticeably impairs or prevents the insertion of the system into narrow vessel radii. A comparison of the flexural rigidities in the distal area of balloon-expandable stent systems (without outer sleeve) with self-expanding stent systems shows that flexural rigidity for self-expanding systems is approximately ten times greater.

In addition, post-dilation with a balloon catheter typically must be carried out after implantation of a self-expandable stent since the radial force of such a stent is often insufficient in practice to fully open the stenosis in the vessel. However, post-dilation has not proven successful in practice, in particular due to the time required, since cardiologists are accustomed to balloon-expanding stent systems, with which the expansion of the stent and the dilation of the vessel are performed in a single step.

Cardiologists would be better served by a self-expandable stent in the crimped state that does not require an outer sleeve. The stent is then dilated with a balloon after positioning at the desired point of the body.

Lau US Patent Publication No. 2009/0234429 describers a self-restraining and expanding endoluminal prosthesis. The self-expanding prosthesis has a plurality of hook elements that protrude transversally from each crosspiece running in the longitudinal direction. In the expanded state, these elements are arranged freely on the lattice of the stent. In order to hold the prosthesis in the compressed state, these elements have to be hooked to one another, wherein the hook elements have to be rotated for this purpose. This can only be implemented manually, and therefore the transition into the compressed state is associated with a high level of effort. It is also disadvantageous that, as a result of the rotation of the hook elements, the pointed ends in particular of the hooks protrude from the outer face of the stent and enlarge the circumference of the system. When introducing the system into the body and passing it through narrow vessel points, the stent or the system may hook onto the vessel wall and injure the vessels. An undesired premature opening of the stent could also be caused as a result.

A balloon-actuated stent with interlocking elements is described in Mathis EP Patent 1,266,638 B1. The stent includes a plurality of bridges on the lattice that interconnect the crosspieces. In the compressed state, each bridge engages in an adjacent bridge, and, in the expanded state, each bridge is spaced from the adjacent bridge. In this case, each bridge at its front end has a widened portion, which extends in the plane of the circumference of the vessel support. For engagement, the crosspieces have corresponding recesses extending in the plane of the circumference. A disadvantage of these connecting elements in the form of bridges is a risk that these connections detach (unclip) from one another when passed through sharp curves, that is to say when the stent is bent, since they only exist in the circumferential plane, thus resulting in the risk that the vessel support will open prematurely.

Steinke et al., U.S. Pat. No. 6,623,521 describes an expandable stent with sliding and locking radial elements. The sliding and locking elements form at least one ratcheting mechanism. The ratcheting mechanism allows the radial elements to slide and thus allows a change in diameter of the stent from the compressed state into the expanded state, but prevents radial recoil from the expanded state. A disadvantage of this solution is that the radial elements are formed in a rib-shaped manner, wherein the ribs run in the circumferential direction of the stent. Due to the length of these ribs, there is a risk in the compressed state that the ribs will protrude outwardly from the stent, thus resulting in a risk of vessel injuries.

Fordenbacher U.S. Pat. No. 5,733,328 describes an expandable stent with sliding members. The stent includes parallel, finger-shaped crosspieces running in the circumferential direction and crosspieces running in the longitudinal direction are provided. The crosspieces running in the circumferential direction have outwardly protruding, convex portions, and the crosspieces running in the longitudinal direction have notches, wherein the convex portions and the notches can latch into one another. As a result, the stent can be fixed with different diameter sizes, wherein the latching mechanism merely counteracts a force in the circumferential direction, but has not proven to be effective in terms of the locking effect with forces in the radial direction, as are produced during the insertion of a stent into the body along vessel curves. Furthermore, there is a risk of injury from outwardly protruding ribs.

### SUMMARY

A preferred embodiment medical implant has a compressed and expanded state. The implant has open-worked, preferably hollow-cylindrical, main structure including a plurality of crosspieces, of which at least two are inter-connected in each case at node elements. At least one first barrier element is provided on a plurality of first node elements and at least one second barrier element is provided on a plurality of second node elements in each case adjacent to a respective first node element. A first barrier element and a respective adjacent second barrier element lock together as the implant transitions from the expanded state into the compressed state to inhibit transition back into the expanded state, such that the barrier opposes a force in the circumferential direction and opposes a force in the radial direction.

A preferred method for making a medical implant having a compressed and expanded state includes producing a starting tube. The starting tube is reshaped to form a profiled tube by pushing through or drawing and with use of a tool comprising a die and a plug. Node elements and crosspieces are cut from the profiled tube by laser beam cutting to provide an open-worked, preferably hollow-cylindrical main structure including a plurality of crosspieces, of which at least two are interconnected in each case at node elements.

A preferred method for making a medical implant having a compressed and expanded state includes producing a starting tube includes producing a starting tube. Node elements and crosspieces are cut from the starting tube by laser beam cutting. A plurality of first barrier elements and a plurality of second barrier elements are separately produced. The first barrier elements and the second barrier elements alternately to nodes adjacent in the circumferential direction. A plurality of first barrier elements and a plurality of second barrier elements are produced on the node elements by selective laser melting.

### DESCRIPTION OF THE DRAWINGS

FIG. 1 shows a cross section through a first exemplary embodiment of an implant according to the invention in the area of first and second node elements,

FIG. 2 shows a cross section through a profiled tube, from which the first exemplary embodiment according to FIG. 1 is produced,

FIG. 3 shows the cross section according to FIG. 2 during the laser beam cutting process,

FIG. 4 shows a perspective view from the side of a first node element with adjoining crosspieces of the exemplary embodiment according to FIG. 1,

FIG. 5 shows a longitudinal section through a tool for the extrusion process with a starting tube arranged therein,

FIG. 6 shows a schematic diagram of a cross section through the tool according to FIG. 5, wherein the tube cross section has no profile,

FIG. 7 shows a further cross section through the tool according to FIG. 5,

FIG. 8 shows a perspective view from the side of a profiled tube,

FIG. 9 shows the cross section according to FIG. 7 and the course of the laser beams during the laser beam cutting process,

FIG. 10A shows a cross section through an implant according to the invention in accordance with the first exemplary embodiment after the laser beam cutting process,

FIGS. 10B to 10D show a perspective view from the side of various details of the implant according to the invention in accordance with the first exemplary embodiment after the laser cutting process in the expanded state,

FIGS. 10E to 10G show a perspective view from the side of various details of the implant according to the invention in accordance with the first exemplary embodiment after the laser cutting process in the compressed state,

FIG. 11 shows cross sections through adjacent node elements in accordance with a second exemplary embodiment of an implant according to the invention after the production of a corresponding semi-finished product without barrier elements,

FIG. 12 shows the cross sections through adjacent node elements according to

FIG. 13 after a further step of the production method with barrier elements in the expanded state,

FIG. 13 shows the node elements according to FIG. 12 in the compressed state,

FIG. 14 shows a cross section through an implant according to the invention in a third exemplary embodiment in the area of the first and second node elements in the compressed state, and

FIG. 15 shows a cross section of the exemplary embodiment according to FIG. 14 during the laser beam cutting of a profiled tube semi-finished product.

### DETAILED DESCRIPTION

The present invention concerns an implant, in particular an intraluminal endoprosthesis with an open-worked, preferably hollow-cylindrical main structure composed of a plurality of crosspieces, which are interconnected at node elements, wherein the implant can assume an expanded state and a compressed state, and also to a method for making an implant of this type. The invention also concerns a system formed of a catheter with balloon and such an implant, and also to a method for making a system of this type.

Embodiments of the invention provide an implant that securely locks the implant in the compressed state without an outer sleeve and which remains securely locked, even during insertion into the body and when passed through narrow vessel points. Furthermore, injury to the vessels caused by an implant during the insertion process can be avoided. Embodiments also provide a corresponding system formed of a catheter and implant, and also a simple and cost-effective production method for an implant of this type and for a system of this type.

A particular preferred embodiment includes at least one first barrier element on the implant at each of a plurality of first node elements, and at least one second barrier element at each of a plurality of second node elements in each case adjacent to a respective first node element in the circumferential direction, wherein a first barrier element and a respective second barrier element adjacent in the circumferential direction in each case lock together or, in other words, latch into one another, as the implant transitions from the expanded state into the compressed state, preferably during crimping, and thus inhibit the transition back into the expanded state, wherein the barrier opposes a force in the circumferential direction and against a force in the radial direction.

An advantage of preferred embodiments of the invention is that the expansion barriers (first barrier element and second barrier element) lock together during crimping and thus prevent independent expansion of the implant, even if the main structure of the stent is produced from a shape-memory alloy, for example consists of nitinol, which performs a temperature-dependent shape conversion. If a specific diameter of the implant is undershot during crimping, the expansion barriers latch and keep the diameter of the implant in the compressed state due to the locked position, even if relatively small forces with components in the radial direction and/or in the circumferential direction act on the implant, for example during insertion into the body. The expansion barriers can be released by expansion with a balloon catheter, which can apply greater forces to the implant. The implant then expands independently.

In this case, the expansion barriers do not extend merely in the circumferential direction as in the prior art, but also have a “third dimension” to a certain extent. As will be explained further below in greater detail, they are formed in such a way that they also block against forces occurring in the radial direction so that the locked position is not opened, for example when passed through narrow curves in vessels during the insertion of the implant into the body. The compressed state is thus assumed reliably and independently of the movement of the implant and is only cancelled by the dilation with the balloon of the catheter. Preferred medical implants and systems of the invention therefore can provide greater reliability in application.

During dilation, all expansion barriers distributed over the implant advantageously have to be released substantially at the same moment in time, that is to say in a short period of time, particularly preferably simultaneously, since, with non-simultaneous release, that is to say non-uniform release, there is a risk of a local super elevation of load at one or more points of the main structure, which may cause the material to break.

The main structure of the implant according to the invention can contain a shape-memory alloy (for example nitinol) or any other suitable material, in particular a metal material. The present invention is therefore not limited to self-expandable implants, in particular self-expandable stents or heart valve stents. Due to the invention however, the handling with a balloon catheter established in the field of medicine, namely the expansion of the implant and the dilation of the vessel in a single step at the point to be treated, for example with a balloon, can also be applied for self-expandable stents or heart valve stents.

In accordance with preferred embodiments of the invention, there are two particular arrangements for combining the above-mentioned first barrier element with a second barrier element such that these form the locked effect according to the invention. The invention is not limited however to the embodiments specified below.

In a first embodiment, the first barrier element is formed as a female element with an undercut, which is open in the circumferential direction and closed in the radial direction, for example as a recess that is spherical at least in some portions and/or cylindrical at least in some portions, and the second barrier element is formed as a complementary male element, for example as a pin that is spherical at least in some portions and/or cylindrical at least in some portions, protruding from the node element in the circumferential direction. In this case, the longitudinal axis of the cylinder forming the cylindrical pin runs parallel to the longitudinal direction of the implant, which runs perpendicular to the circumferential direction and perpendicular to the radial direction. The feature that the female element is open in the circumferential direction particularly preferably indicates that a centerline extending from the center of the female element and through the center of the opening runs approximately in the circumferential direction. It is also advantageously necessary for materials that are not resilient or that are only slightly resilient for the maximum diameter of the male element in the radial direction to be greater than the diameter of the opening and smaller than the maximum inner diameter of the female element in the radial direction. With use of materials having a high level of resilience, such as nitinol, an identical to larger maximum diameter of the male element in the radial direction compared to the inner diameter of the female element in the radial direction can also be used. Due to the specified size ratios, it is ensured that the male element locks behind the undercut and also that the male element can be received by the female element. In the compressed state, the male element latches into the female element, in particular behind the undercut of the female element.

Alternatively to the design of the female and male element with a round cross section, other cross-sectional shapes, for example the shape of a triangle or square or the like, which are complementary in each case, can be used. The male element is preferably fastened to the respective second node element via a short neck.

This exemplary embodiment entails a simple solution for ensuring that the barrier element together with the second barrier element opposes a force in the circumferential direction and against a force in the radial direction. The undercut opposes a force exerted in the circumferential direction, and the walls of the female element arranged in the direction of the respective force, for example the concave walls of the female element arranged in the direction of the respective force, block against a force exerted in the radial direction.

In a second embodiment, two similar barrier elements latch into one another. In this case, the first barrier element is formed as an element that is U-shaped in cross section with a first opening in a first radial direction and with an undercut arranged at the first opening, and the second barrier element is likewise formed as an element that is U-shaped in cross section with a second opening in a second direction, opposed to the first radial direction, and with an undercut arranged at the second opening. The openings preferably point in the radial direction.

Due to the arrangement of the openings in the first barrier element and in the second barrier element in such a way that said barrier elements are each oriented in the radial direction, it is not necessary to bend elements such that they protrude from the outer face of the implant, as is the case for example in document US 2009/0234429 A1. They can latch directly into one another during crimping, and, together with the undercuts, form a barrier both in the radial direction and in the circumferential direction, more specifically in both opposed directions. In the event of dilation by a balloon, this locked effect can also be released again in a simple manner by bending up these barrier elements. The risk of injury when passing through vessels is thus minimal.

It is also advantageous if the extension of the second barrier element in the radial direction is smaller than the extension of the respective adjacent node element in the radial direction. It is thus ensured, as a result of the barrier element that is only fastened for example to the node element by a short neck, that said barrier element does not protrude from the implant in the radial direction. The risk of injury caused by an implant of this type, which is arranged in a body part or is passed through a body part, for example a vessel, is reduced compared to the solutions according to the prior art.

The length of the neck, at which a second barrier element is fastened to the respective second node element, is preferably less than half a crosspiece diameter. As a result, the barrier element is also prevented from protruding outwardly from the implant, therefore likewise minimizing the risk of injury.

Two first barrier elements are preferably arranged laterally on each first node element, that is to say a first barrier element is preferably arranged in both directions in the circumferential direction. Accordingly, two second barrier elements are arranged laterally on each second node element, that is to say a second barrier element is arranged in both directions in the circumferential direction.

As will be explained in greater detail below, an implant according to the invention can be produced particularly easily and cost-effectively based on a profiled tube. It is accordingly advantageous for the first barrier element and the second barrier element to be formed in each case from a (accordingly shaped) portion of a profile extending parallel to the longitudinal axis of the implant. For this reason, it is likewise advantageous for the crosspieces to also be formed from further portions of profiles extending parallel to the longitudinal axis of the implant.

A preferred embodiment system is formed of an implant and a catheter with a balloon, wherein the implant, which has the above-described features, is crimped onto the balloon of the catheter.

A preferred method for making an implant, in particular the above-described implant, has the following steps:
producing a starting tube, preferably through casting, rapid prototyping or sintering and possibly core hole drilling,
reshaping the starting tube to form a profiled tube through pushing through or drawing and with use of a tool comprising a die and a plug, preferably through extrusion,
cutting out the node elements and the crosspieces from the profiled tube through laser beam cutting, wherein the laser beams preferably run substantially in the radial direction.

An advantage of the production method according to the invention lies in the fact that the desired shape of the barrier elements is already formed by profiling of the starting tube through pushing through or drawing. Only the node elements with the barrier elements and the crosspieces then have to be cut out through laser beam cutting, preferably through radial cuts that are very easily implemented, the light source being arranged inside or outside the implant.

During the reshaping process, the tube is profiled using the die on the outer face of the tube and using the plug on the inner face. The plug is therefore also referred to as a mandrel. The tool is usually heated and rotatably mounted.

Alternatively to the above method, the implant according to the invention can also be formed directly through rapid prototyping. Rapid prototyping is a simple method that is known in principle for the production of complex geometries, but which is not intended for use on a mass scale.

As a further alternative, after production of a starting tube, preferably through casting or sintering or rapid prototyping and possibly core hole drilling, the node elements and the crosspieces of the main structure are cut out from the starting tube through laser beam cutting, preferably using cuts running substantially in the radial direction, that is to say cutting with a laser beam running in the radial direction. The closure system can be produced in parallel, for example from a welding wire or welding strip. The closure system consisting of a plurality of first barrier elements and second barrier elements is then attached alternately to node elements adjacent in the circumferential direction, preferably through adhesive bonding or welding or powder injection molding (PIM) or machining (for example drilling, electric discharge machining or wire electric discharge machining) This method is advantageous in particular if different closure systems are to be arranged on the node elements of the implant. Alternatively, the barrier elements can be produced and connected to the node elements through selective laser melting or laser sintering.

Advantages are also achieved by a preferred method for making a system formed of a catheter with balloon and an implant, wherein the implant is produced by the above-specified method and is then arranged on the balloon of the catheter and is crimped onto.

Preferred embodiments of the invention will now be discussed with respect to the drawings. The drawings may include schematic representations, which will be understood by artisans in view of the general knowledge in the art and the description that follows. Features may be exaggerated in the drawings for emphasis, and features may not be to scale.

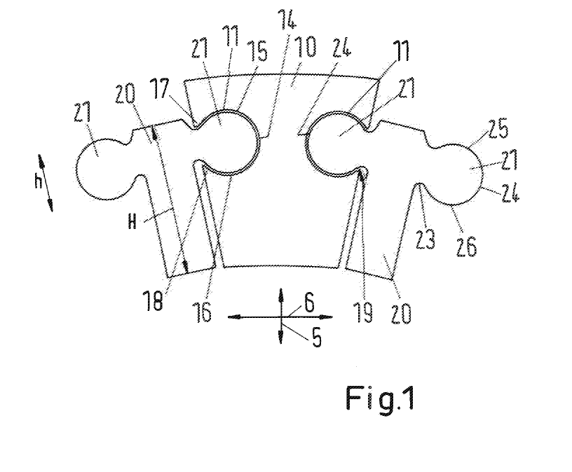

A stent, which is composed of a hollow-cylindrical or tubular main body with a plurality of crosspieces 8 (see FIG. 4), will be described hereinafter on the basis of FIGS. 1 to 10G as a first exemplary embodiment of an implant according to the invention. The crosspieces 8 are arranged substantially in a zigzagged manner in the circumferential direction 6 and run substantially at an incline to the longitudinal axis (longitudinal direction) of the stent. In each case, at least two crosspieces 8 adjoin first node elements 10 and second node elements 20, wherein a first node element 10 and a second node element 20 are provided alternately in the circumferential direction 6.

The longitudinal direction of the stent runs perpendicular to the plane shown in FIG. 1 and perpendicular to the radial direction 5 and to the circumferential direction 6.

Two cylindrical recesses 11 are arranged laterally, that is to say in the circumferential direction, in the first node element 10 and each represent a first barrier element. In the compressed state of the stent shown in FIG. 1, a substantially cylindrical pin 21 is arranged as a second barrier element in each recess 11 and is fastened to the second node element 20 via a neck 23. The diameter h of the pin 21 is slightly smaller than the inner diameter of the recess 11, such that the recess 11 can receive the pin 21 in the compressed state of the stent, as shown in FIG. 1.

The recess 11 has concave wall portions 14, 15 and 16. Accordingly, a convex end portion 24 and further convex portions 25, 26, in each case adjacent to the end portion 24 in the direction of the neck 23, are provided on the pin 21 at the end opposite the second node element 20.

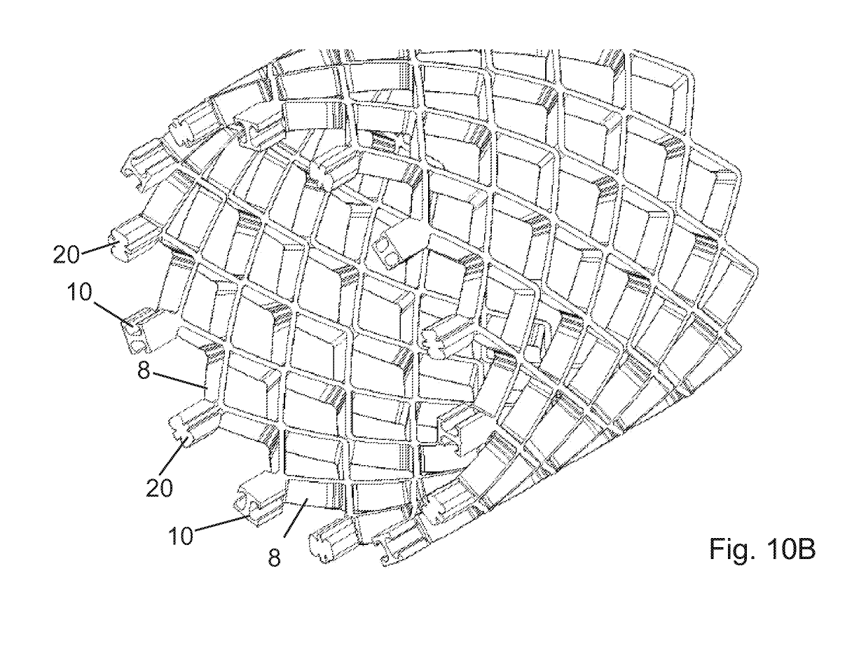

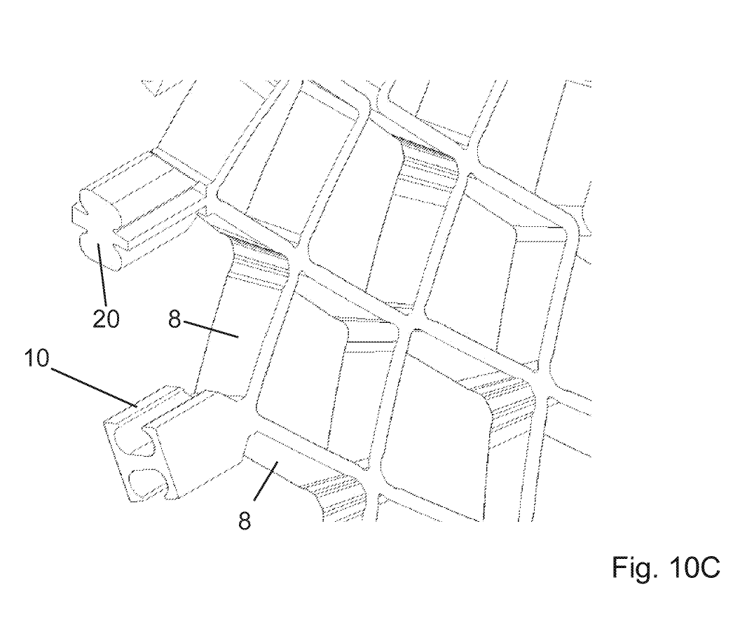

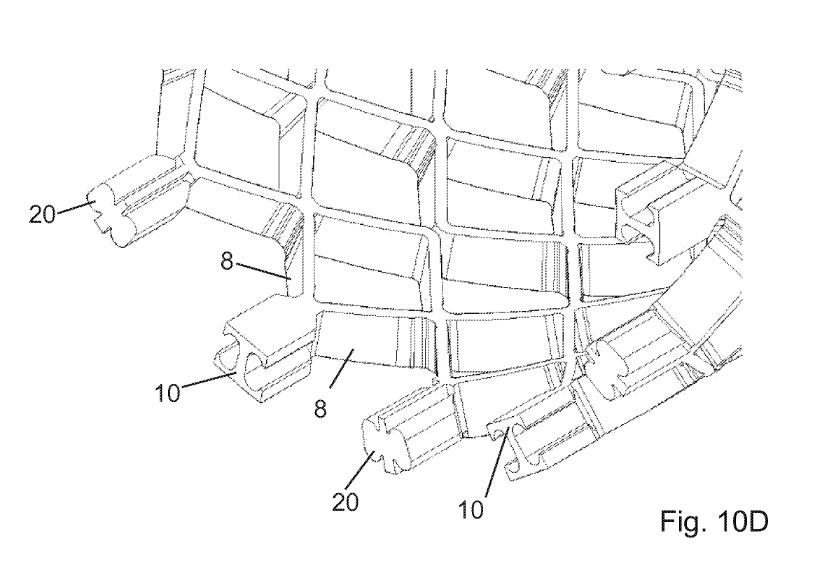

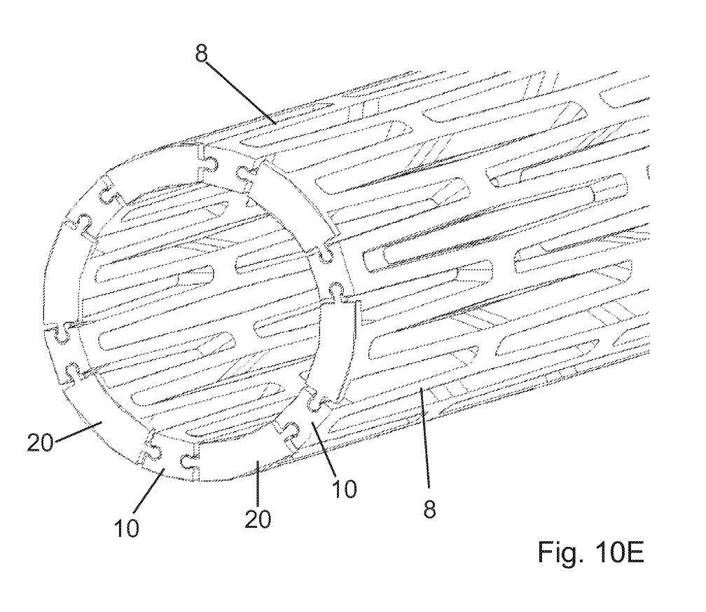

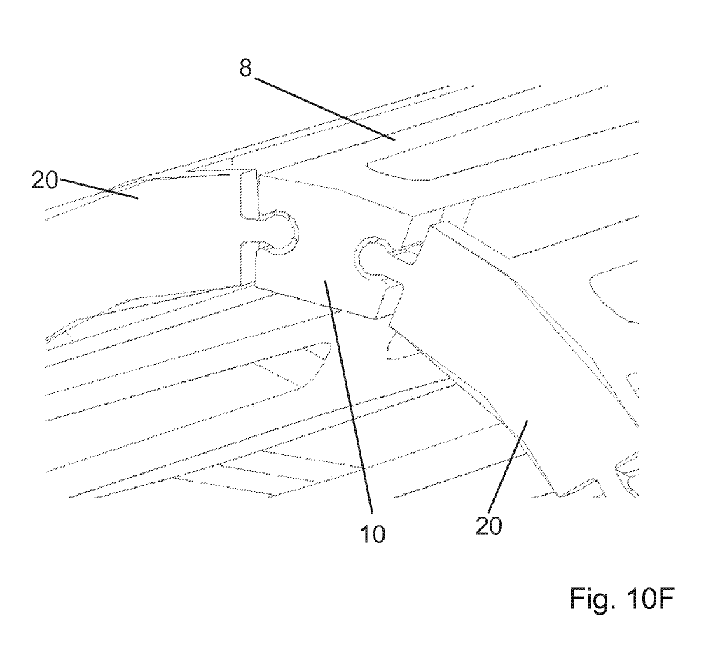

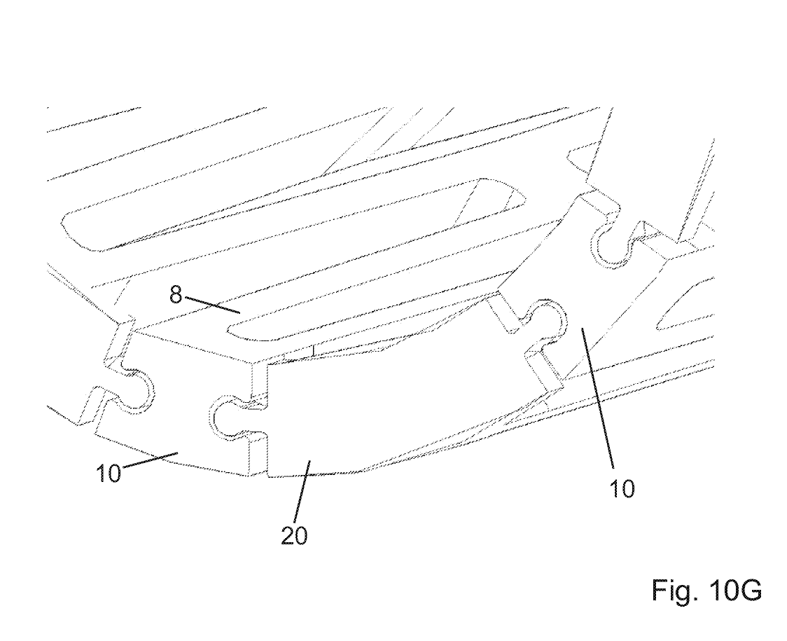

In this exemplary embodiment, each recess 11 has a lateral (running in the circumferential direction 6) opening (aperture) 19. Such an opening 19 can be seen clearly in particular in FIG. 4. The opening 19 is used for the insertion of the pin 21 into the recess 11 during the latching process when the stent transitions from the expanded state (see FIGS. 10A to 10D) into the compressed state (see FIGS. 10E to 10G).

The concave wall portions 15, 16 of the recess 11, which are arranged in the radial direction 5, extend so far in the direction of the opening 19 that they form undercuts 17, 18 via their tips arranged farthest in the direction of the opening 19. The undercuts 17, 18 mean that the pin 21 is held securely by the recess 11 (up to a specific maximum force) or remains latched in the recess 11, even with a force in the circumferential direction 6 occurring during the handling of the stent in the compressed state, said force pulling the first node element 10 and the second node element 20 away from one another.

The height h of the pin 21 in the radial direction 5 is much smaller than the height H of the second node element 20 in the same direction. As a result, the pin 21 does not project or protrude outwardly from the stent. There is thus no risk of injury.

The production method for a stent in accordance with the first exemplary embodiment will be described hereinafter.

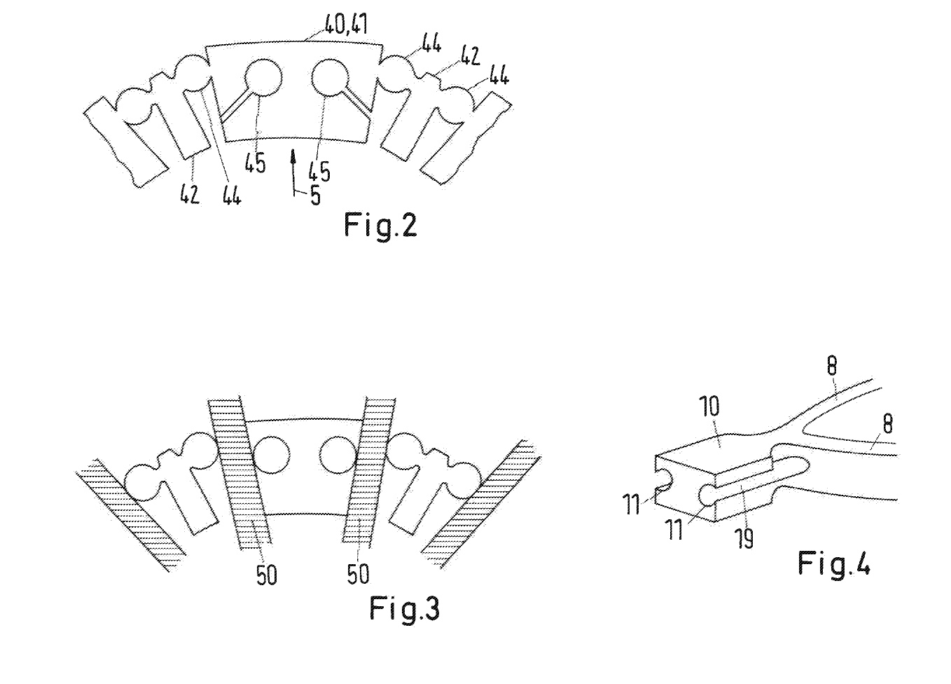

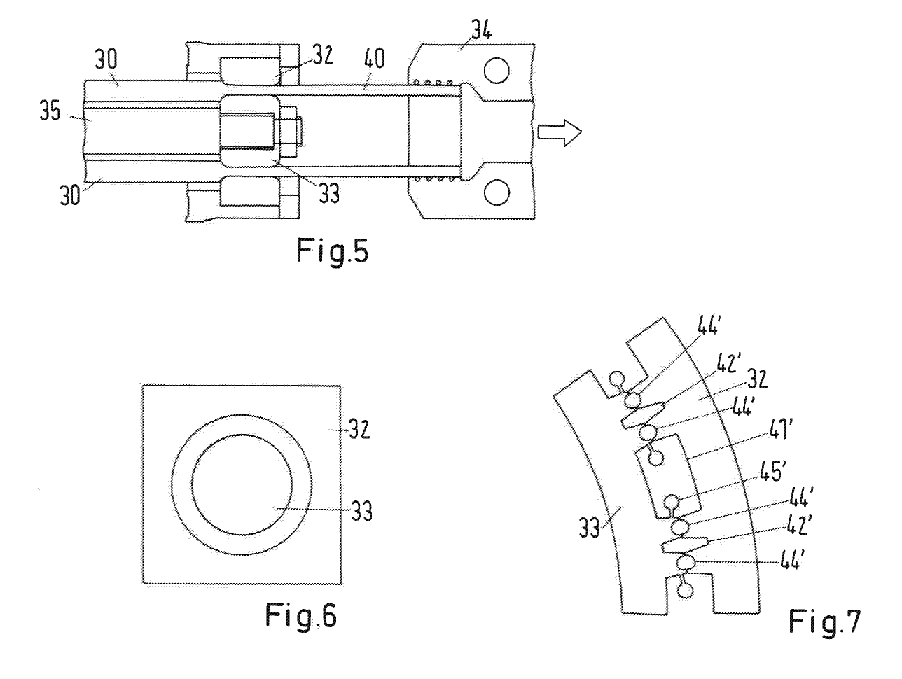

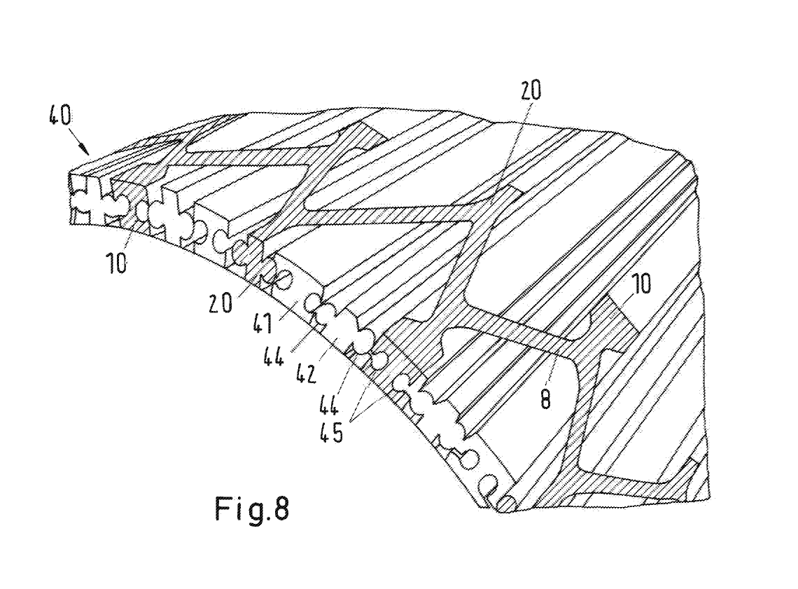

A starting tube 30, for example made of nitinol (alternatively consisting of stainless steel, titanium, a titanium alloy, a cobalt/chromium alloy, a nickel-based alloy, a copper alloy, or a magnesium alloy) is first produced through casting or sintering and possible subsequent core hole drilling. This starting tube is then reshaped via extrusion, which is illustrated in FIG. 5, to form a profiled tube 40, which is shown in FIGS. 2 and 8. For this purpose, the starting tube 30 is introduced into a tool consisting of a die 32 and a plug or mandrel 33 and is drawn through preferably at increased temperature, for example at a temperature up to 200° C. at most, using a drawing tong 34, in which the front end of the profile tube 40 is fixed, wherein the die 32 further preferably is heated before the reshaping process in order to improve the sliding properties. A lubricant is particularly preferably used during the reshaping step. The plug 33 is fastened in this case to a pull rod 35. A profile that has a reduced wall thickness compared to the starting tube 30 is introduced into the starting tube 30 as a result of the extrusion process.

The concentric arrangement of the die 32 and plug 33 is illustrated merely schematically in FIG. 6. The profiling of the die 32 and plug 33 is not shown in this illustration. It can be seen from FIG. 7 that the outer face of the plug 33 and the inner face of the die 32 together form a complementary profile, in each case with recesses 41′, 42′ and 44′, which is used to form crosspieces 41, 42 running in the longitudinal direction and cylinders 44 arranged therebetween and running in the longitudinal direction. Furthermore, the mandrel 33 has pins 45′, which produce substantially cylindrical recesses 45, likewise running in the longitudinal direction, in the profiled tube 40.

The profiled tube 40 formed by the die 32 and plug 33 is therefore composed of crosspieces 41, 42, which run in the longitudinal direction, are arranged in an alternating manner, and are each interconnected via a cylinder 44. The first crosspiece 41 has two substantially cylindrical recesses 45, likewise extending in the longitudinal direction.

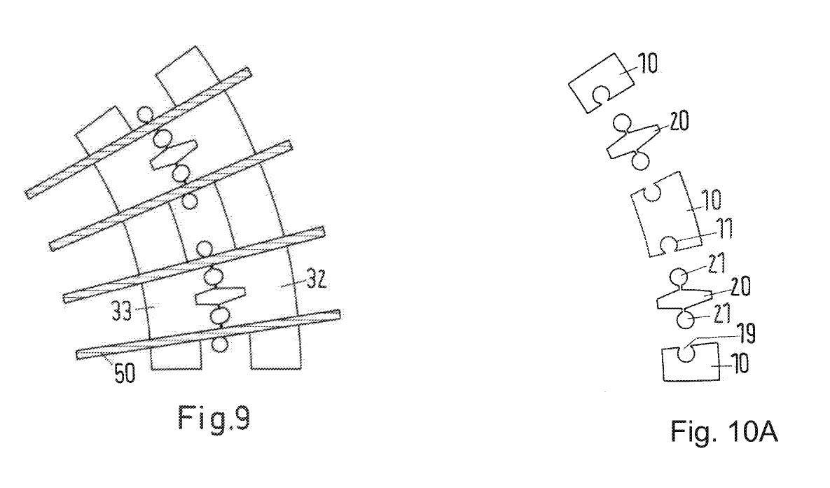

The profiled tube 40 thus produced is worked in the next step through laser beam cutting in order to cut out the crosspieces 8 and the node elements 10, 20 with the barrier elements 11, 21. The beams of the laser running in the radial direction 5 are provided in FIGS. 3 and 9 with the reference sign 50 and in these drawings show the beam course in the area of the node element 10, 20. The laser beams 50 remove material laterally in the area of the crosspieces 41 from the recesses 45 in such a way that the first node elements 10 with the recesses 11 and lateral opening 19 are produced from the crosspieces 41, and the second node elements 20 are produced from the crosspieces 42, and the pins 21 are produced from the cylinders 44.

As can be deduced from FIG. 8, the laser beams 50 are guided between the node elements 10, 20, which are arranged in succession in the longitudinal direction, in such a way that the crosspieces 8 are then cut out from the structure of the profiled tube 40 at an incline relative to the longitudinal direction. The crosspieces 8 converge in the longitudinal direction to the respective other node element 20, 10, etc. The structure cut out from a profiled tube 40 using a laser beam is illustrated in a hatched manner in FIG. 8.

After the laser beam cutting process, the stent is optionally subjected to post-treatment steps, such as deflashing, etching and electropolishing, and is then finished. In particular, flushing or irregularities over the surface of the stent possibly caused by the laser beam cutting process are removed during the post-treatment. The stent according to the invention with first barrier elements in the form of cylindrical recesses 11 and second barrier elements in the form of pins 21 can then be crimped onto the balloon of a catheter and can thus be transferred from the expanded state (see FIGS. 10A to 10D) into the compressed state (see FIGS. 10E to 10G). Here, the pins 21 latch into the respective adjacent recess 11 and thus lock the stent according to the invention, even after removal of the crimping tool, in the compressed state.

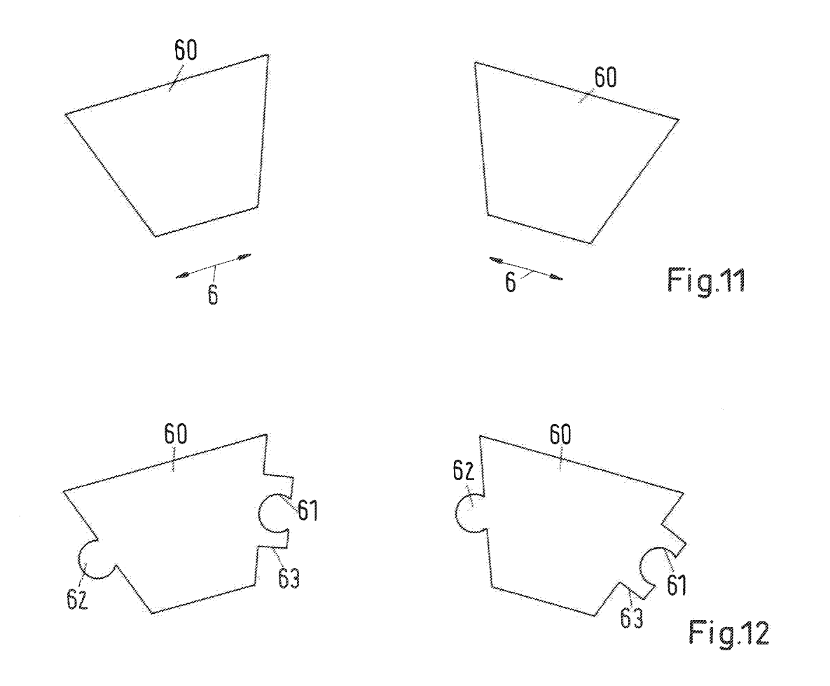

FIGS. 11 to 13 show a modification of the first exemplary embodiment of a stent according to the invention, which has a first barrier element in the form of a cylindrical recess 61 on node elements 60 on one side, and has a second barrier element in the form of a cylindrical pin 62 on the second side, said barrier elements each extending in the circumferential direction 6. Here, the recess 61 and the pins 62 are arranged on adjacent node elements 60 in such a way that they are arranged opposite one another, as shown in FIG. 12.

Compared to the first exemplary embodiment, two different barrier elements 61, 62 are arranged on each node element 60 in the second exemplary embodiment, whereas, with the first exemplary embodiment, two similar barrier elements (either two recesses 11 or two pins 21) are provided on each node element 10, 20. Alternatively, different barrier elements can of course also be arranged on each node element with the first exemplary embodiment, or similar barrier elements can be arranged on each node element with the second exemplary embodiment.

The recess 61 of the second exemplary embodiment is designed similarly to the recess 11 of the exemplary embodiment illustrated in FIGS. 1 to 10G. In particular, the recess 61 also has undercuts in addition to an insertion opening for the pin 62. However, the first barrier element 61 protrudes from the respective node element 60 in a substantially cuboidal protrusion or shoulder 63, whereas the first barrier element 11 of the first exemplary embodiment is arranged within the node 10, without a separate shoulder.

The pin 62 corresponds in terms of design to the pin 21 of the first exemplary embodiment illustrated in FIGS. 1 to 10G.

The second exemplary embodiment illustrated in FIGS. 11 to 13 can be produced by working a starting tube produced similarly to the first exemplary embodiment from a material through laser beam cutting, similarly to the first exemplary embodiment. Here, the laser beam merely cuts the main structure from the starting tube consisting of the crosspieces 8 and the nodes 60. The reshaping step is omitted.

A material, for example nitinol, is then applied in a thin layer in powder form to the stent structure thus produced.

For the production of the powder, the solid material is first atomized, mechanically size-reduced or chemically separated in solution. The materials used for the laser melting process are mass-produced materials, which do not contain any binder. For example, the materials comprise stainless steel, tool steel, aluminum, aluminum alloys, titanium, titanium alloys, cobalt-chromium, nickel-based alloys, copper alloys, ceramic, plastics.

This powdery material is fully melted locally using a laser beam or an electron beam in the area of the barrier elements 61, 62 and then solidifies again. After solidification, it forms a solid material layer, specifically the barrier elements 61, 62. Here, the barrier elements 61, 62 can be produced in a number of steps, wherein a layer of the respective barrier element 61, 62 is finished in each step.

For the method of selective laser melting or selective laser sintering or rapid prototyping, the data for the guidance of the laser beam are produced from a 3D-CAD body via software.

Elements manufactured with selective laser melting are characterized by a high density. This ensures that the mechanical properties of the generatively produced element largely correspond to those of the base material.

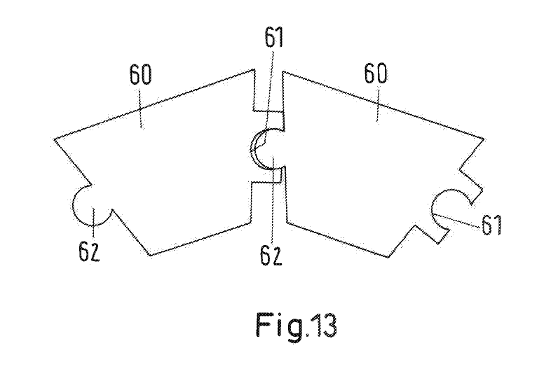

The finished component part is then cleaned of excess powder, is finished for example via post-treatment steps, such as deflashing, etching and electropolishing, and is optionally crimped onto the balloon of a catheter similarly to the first exemplary embodiment. During the crimping process, the pins 62, as illustrated in FIG. 13, latch into the recesses 61.

Alternatively, the barrier elements may also be formed for example by a welding wire and welded or adhesively bonded onto the node elements 60.

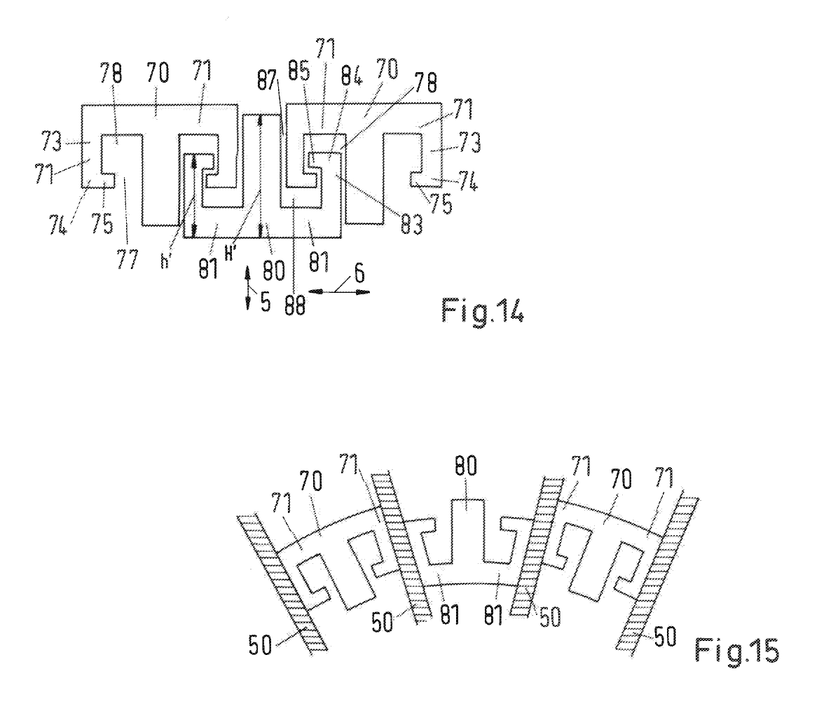

Lastly, FIGS. 14 and 15 show a third exemplary embodiment of an implant according to the invention. Here, node elements 70, 80 are formed at the ends of the crosspieces, wherein the first node element 70 in each case has two first barrier elements 71, and the second node element 80 in each case has two second barrier elements 81, in each case on either side in the circumferential direction.

The first barrier element 71 comprises a U-shaped crosspiece arrangement with an opening 77 provided in the radial direction 5. The crosspiece arrangement surrounds a recess 78, which is rectangular in cross section. Here, one branch of the U is formed by the node element 70, and the other branch of the U is formed by a crosspiece 73 running in the radial direction 5. A further crosspiece element 75 is arranged in the circumferential direction at the front end 74 of the crosspiece 73 pointing inwardly with respect to the stent and extends in the circumferential direction 6, but still leaves open the opening 77 of the U in the radial direction 5. The crosspiece element 75 forms an undercut.

The second barrier element 81 is designed in a manner complimentary to the first barrier element 71. The second barrier element 81 also forms a U-shape with an opening 87 extending in the radial direction 5, said opening being directed however opposite to the opening 77 in the first barrier element 71, and with a recess 88 surrounded by the U. Furthermore, a lateral crosspiece 83 and a further crosspiece element 85 extending in the circumferential direction 6 are arranged at the front end 84, said crosspiece element only partly closing the opening 87 however and forming an undercut.

During crimping, the first barrier element 71 locks with the second barrier element 81, as shown in FIG. 14. In particular, the front end 74 with the transversely running crosspiece element 75 of the first barrier element 71 is received by the recess 88 of the second barrier element 81, and, conversely, the front end 84 of the crosspiece 83 with the transversely running crosspiece element 85 is received by the recess 78 of the first barrier element 71. If forces then occur in the circumferential direction 6 as a result of the movement of the stent in the compressed state, these forces are thus received by the node elements 70, 80 and the crosspieces 73, 83 of the barrier elements 71, 81. With forces in the radial direction 5, these forces are received by the crosspiece elements 75, 85 forming the undercuts or by the middle parts of the U-shape of the barrier elements 71, 81 arranged between the crosspiece 73 and the node element 70 and also between the crosspiece 83 and the node element 80.

Similarly to the first exemplary embodiment, the extension h′ of the second barrier element 81 is smaller than the extension H′ of the adjacent second node element 80, in each case in the radial direction 5, so that injury is avoided.

FIG. 15 shows a step during the production of an implant of this type. Similarly to the procedure with the first exemplary embodiment, with this exemplary embodiment a profiled tube can first be produced via extrusion from a starting tube, and the required structures, in particular the crosspieces, node elements and barrier elements, can then be cut out from the profiled tube using laser beam cutting by radially running laser beams 50 (see FIG. 15). The implant may optionally then be postprocessed and crimped onto the balloon of a catheter, similarly to the exemplary embodiments described previously.

The above-illustrated solutions according to the invention utilize expansion barriers consisting of a first barrier element and a second barrier element that act not only in the circumferential plane of the implant, but also in the radial direction. As a result, the compressed state can be reliably maintained, even with a wide range of movements, which are performed by such an implant, for example when inserted into the body or when passed through a vessel. The expansion barriers are located over the length of the stent and are distributed over the circumference of the implant. The expansion barriers latch during the crimping process and thus prevent independent expansion of the stent. Due to the greater forces applied from dilation of the balloon, the locked positions of the expansion barriers can be released again such that the implant can then expand at the treatment location, either independently or due to the dilation forces of the balloon.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

LIST OF REFERENCE NUMERALS

| | |
|---|---|
| 5 | radial direction |
| 6 | circumferential direction |
| 8 | crosspiece |
| 10 | first node element |
| 11 | recess |
| 14, 15, 16 | concave portion |
| 17, 18 | undercut |
| 19 | opening |
| 20 | second node element |
| 21 | pin |
| 23 | neck |
| 24, 25, 26 | convex portion |
| 30 | starting tube |
| 32 | die |
| 33 | plug, mandrel |
| 34 | drawing tong |
| 35 | pull rod |
| 40 | profiled tube |
| 41, 42 | crosspiece |
| 44 | cylinder |
| 45 | recess |
| 50 | laser beam |
| 60 | node element |
| 61 | recess |
| 62 | pin |
| 63 | shoulder |
| 70 | node element |
| 71 | first barrier element |
| 73 | crosspiece |
| 74 | front end of the crosspiece 73 |
| 75 | crosspiece element |
| 77 | opening |
| 78 | recess |
| 80 | second node element |
| 81 | second barrier element |
| 83 | crosspiece |
| 84 | front end of the crosspiece 83 |
| 85 | crosspiece element |
| 87 | opening |
| 88 | recess |
| 41′ | recess |
| 42′ | recess |
| 44′ | recess |
| 45′ | pin |
| h | height of the pin 21 |
| h′ | height of the barrier element 81 |
| H, H′ | height of the node element |

1. A medical implant that has a compressed and expanded state comprising:
an open-worked main structure including a plurality of crosspieces, of which at least two are interconnected in each case at node elements;
at least one first barrier element provided on a plurality of first node elements and at least one second barrier element provided on a plurality of second node elements in each case adjacent to a respective first node element;
wherein a first barrier element and a respective adjacent second barrier element lock together as the implant transitions from the expanded state into the compressed state to inhibit transition back into the expanded state, wherein the barrier opposes a force in the circumferential direction and opposes a force in the radial direction.

2. The implant as claimed in claim 1, wherein the main structure is hollow-cylindrical.

3. The implant as claimed in claim 1, wherein the first barrier element comprises a female element, which is open in the circumferential direction, with an undercut, and the second barrier element comprises a male element.

4. The implant as claimed in claim 3, wherein the undercut and comprises a recess that is spherical and/or cylindrical at least in some portions and the male element comprises a pin having a complementary shape.

5. The implant as claimed in claim 1, wherein the first barrier element is U-shaped in cross section with a first opening in a first radial direction and an undercut arranged at the first opening, and the second barrier element is is U-shaped in cross section with a second opening in a second direction, opposed to the first radial direction, and also with an undercut arranged at the second opening

6. The implant as claimed in claim 1, wherein extension of the second barrier element in the radial direction is smaller than extension of the respective adjacent second node element in the radial direction.

7. The implant as claimed in claim 1, wherein the first barrier element and the second barrier element are each formed from an accordingly shaped portion of a profile extending parallel to the longitudinal axis of the implant.

8. The implant as claimed in claim 5, wherein the crosspieces are formed from portions of profiles extending parallel to the longitudinal axis of the implant.

9. A system formed of an implant as claimed in claim 1 and a catheter with a balloon, wherein the implant is crimped onto the balloon of the catheter.

10. A method for making a medical implant having a compressed and expanded state:
producing a starting tube;
reshaping the starting tube to form a profiled tube by pushing through or drawing and with use of a tool comprising a die and a plug,
cutting node elements and crosspieces from the profiled tube by laser beam cutting to provide an open-worked main structure including a plurality of crosspieces, of which at least two are interconnected in each case at node elements.

11. A method as claimed in claim 10, wherein said producing comprises casting, sintering, rapid prototyping and/or core hole drilling.

12. A method as claimed in claim 10, wherein said cutting comprises cutting with laser beams in the radial direction.

13. A method as claimed in claim 10, further comprising arranging the implant on balloon of a catheter and is then crimping the implant thereto.

14. A method as claimed in claim 10, wherein the main structure is hollow-cylindrical.

15. A method for making a medical implant having a compressed and expanded state:
producing a starting tube;
cutting out node elements and crosspieces from the starting tube by laser beam cutting;
separately producing a plurality of first barrier elements and a plurality of second barrier elements and attaching the first barrier elements and the second barrier elements alternately to nodes adjacent in the circumferential direction; and
producing a plurality of first barrier elements and a plurality of second barrier elements on the node elements by selective laser melting.

16. A method as claimed in claim 15, wherein said producing a starting tube comprises casting, sintering, rapid prototyping and/or core hole drilling.

17. A method as claimed in claim 15, wherein said separately producing comprises adhesive bonding, welding, powder injection molding, or machining.

18. A method as claimed in claim 15, further comprising arranging the implant on balloon of a catheter and is then crimping the implant thereto.
